(19) United States
(12) Patent Application Publication
(10) Pub. No.: US 2007/0142749 A1
(43) Pub. Date: Jun. 21, 2007

(54) Apparatus for medical and/or simulation procedures

(76) Inventors: Oussama Khatib, Palo Alto, CA (US); Xin Ma, Singapore (SG); Wieslaw Lucjan Nowinski, Singapore (SG); Marcelo Huibonhoa Ang JR., Singapore (SG); Chee Kong Chui, Singapore (SG); Zi Rui Li, Singapore (SG)

Correspondence Address: DICKSTEIN SHAPIRO LLP, 1825 EYE STREET NW, Washington, DC, 20006-5403, US

(21) Appl. No.: 10/590,944

(22) PCT Filed: Mar. 1, 2005
(86) PCT No.: PCT/SG05/00063

§ 371 Date: Aug. 28, 2006

(30) Foreign Application Priority Data
Mar. 4, 2004 (SG) 200401086-4

(51) Int. Cl. A61B 5/103

(52) U.S. Cl. 600/587

(57) ABSTRACT

In a preferred embodiment, medical apparatus comprising a catheter (12) which operable to be inserted into a human subject (10) is disclosed herein. Haptic sensors (22, 24) and deformation sensors (26, 27) are connected to the catheter (12) and these are disposed at a plurality of locations along the length of the catheter (12). The haptic sensors measures 3D forces (18) acting on the catheter (12) at the disposed locations and these forces are provided to a haptic feedback device (14) which provides haptic feedback to, an interventional radiologist. The deformation sensors (26, 27) on the other hand, measures the deformation of the catheter (12) at the disposed locations and this information determines the shape of the catheter (12) which is represented on a display (16) for viewing by the radiologist.

### BACKGROUND AND FIELD OF THE INVENTION

This invention relates to apparatus for medical and/or simulation procedures for interventional medicine.

In invasive medical procedures such as interventional radiology, interventional cardiology and interventional neuroradiology, an interventional medical specialist (radiologist, cardiologist or neuroradiologist) needs to place a catheter at a target site in a patient by introducing the catheter through a blood vessel. Diagnostic images of the patient are taken before a procedure to provide the specialist with images of the blood vessel's structure. During the intervention procedure, the specialist relies mainly on the X-ray images and hand and eye coordination to navigate and position the catheter at the target site and the procedure requires considerable skill and the development of haptic “feel”, which comes with experience, so that the specialist can interpret feedback through the catheter to the specialist's hand of resistance to movement for example, that might be an indication of an obstruction and/or pressure being applied by the tip of the catheter to the blood vessel which might lead to the blood vessel being ruptured. To avoid rupturing the blood vessel, when there is an indication of an obstruction, the specialist usually takes more X-ray images to ascertain the structure of the blood vessel and thus the patient and interventional staff are subjected to more radiation, which should be avoided.

It is an object of the invention to provide medical apparatus which can assist the specialist in such a procedure.

It is another object of the invention to provide medical apparatus which can be used in a simulation procedure for training of such specialists.

### SUMMARY OF THE INVENTION

In a first aspect of the invention, there is provided medical apparatus comprising an elongate intervention device being operable to be inserted into a human or animal subject, and at least one force-measuring sensor connected to the intervention device and disposed at least one location along the intervention device; the at least one force-measuring sensor being arranged to measure 3-D forces acting on the intervention device at the disposed locations.

Preferably, a force-measuring sensor is disposed near or at the insertion end of the intervention device. This measures the force acting on the tip of the device as the device is inserted into the subject and maneuvered to the target site.

Preferably, a plurality of force measuring sensors is provided and disposed at different locations along the intervention device. The force-measuring sensors may be disposed at intervals, which may be uniform, along the whole or a part of the length of the intervention device. At each location, a plurality of coplanar sensors may be provided. Each of these sensors measures the friction force between the intervention device and vascular walls of the human subject.

Preferably, the medical apparatus further comprises at least one deformation sensor connected to the intervention device and disposed at least one location along the length for measuring the deformation of the intervention device at the disposed location. The deformation information can be used to determine the shape of the intervention device. Similar to the force-measuring sensors, at each location a plurality of coplanar sensors may be provided.

Preferably, the deformation sensors and the force-measuring sensors are interleaved at intervals with each other along the length direction of the intervention device. The sensors may be interleaved at uniform intervals. The deformation sensors may be fibre optic sensors and the force-measuring sensors may be haptic sensors. If the intervention device is a catheter, then the sensors are connected to the catheter. Alternatively, the intervention device may be a guide wire for a catheter and the sensors are connected to the guide wire.

In a second aspect of the invention there is provided medical apparatus comprising an elongate intervention device being operable to be inserted into a human or animal subject; and at least one deformation sensor connected to the intervention device and disposed at least one location along the length direction of the intervention device, the at least one deformation sensor being arranged to measure the deformation of the intervention device at the disposed location. The deformation information can be used to determine the shape of the intervention device.

The apparatus is preferably connected to a processing means which determines, from the sensors, details of the shape of and/or forces on the catheter/lead wire and displays the details and/or provides haptic feedback to the specialist. The processing means may further comprise means for displaying the shape of the intervention device.

According to a third aspect of the invention, there is provided haptic feedback apparatus comprising force application means arranged to apply a force to an elongate intervention device, control means arranged to control the force applied to the intervention device by the force application means, the control means being connected to at least one sensor arranged to sense a remote force on the intervention device and the control means being arranged to calculate the applied force in accordance with the remote force, the applied force being an amplification of the remote force.

Preferably, the force application means applies both an axial and a radial force to the catheter. The force application means may comprise a resilient member arranged to apply the said force to the intervention device. Preferably, the haptic feedback apparatus further comprises a sensor arranged to detect fictional force between the resilient member and the intervention device. The detected frictional force may then be used to control the amount of applied force.

Preferably, the haptic feedback apparatus further comprises means for tracking the rotational and/or linear movement of the intervention device. Specifically, the tracking means may be in the form of a wheeled encoder.

The haptic feedback apparatus may further comprise means for comparing the remote force with a reference force. This may be used for training purposes under simulation environment since the comparison can be used to determine the catheterisation skills of an interventional radiologist. For example, if the reference force is the force that will rupture a blood vessel and if the detected remote force is more than the reference force, this would mean that the radiologist would have ruptured a blood vessel if the procedure is real. In this case, the intervention device may be operable to be inserted into a simulated human model and the remote force may be generated using computer simulation.

Alternatively, in a real procedure, the intervention device is operable to be inserted into a human subject.

The at least one sensor may be disposed near or at a tip of the intervention device. Preferably, the control means is connected to a plurality of sensors disposed along the length of the intervention device.

This invention also includes a method of using the medical apparatus in accordance with the first aspect and/or the second aspect of the invention and/or the haptic feedback apparatus in an interventional or simulated interventional procedure.

The described embodiment is for particular use in augmented interventional radiology in which an interventional device such as the catheter passes first through the haptic feedback apparatus and then is inserted into a blood vessel of a patient in a traditional way. The sensors transmit the signals back to the apparatus which magnifies the forces accordingly. This increases the accuracy of the interventional procedure. Furthermore, the force and other sensed information can be processed such that the tip of the catheter can be displayed in real time with respect to the blood vessel so that direction of the catheter's tip can be monitored. The described embodiment of the invention is also applicable as an interventional radiology simulator in which the forces from the sensors can be calculated from a physical and realistic model when using an interventional radiological simulator. The apparatus can then receive control signals based on the simulation for training purposes and to understand catheter-blood vessel interaction. In addition, information about the shape and location of the catheter can be used to guide model-data registration.

These approaches both real and simulated are also applicable for remote operations.

The actions of an interventional radiologist or other specialists can be also be measured by the sensors and compared to standard actions, for skill assessments and similarly a simulation system can be validated by measuring forces and torques produced by it in comparison to real life data.

### BRIEF DESCRIPTION OF THE DRAWINGS

Embodiments of the invention will now be described, by way of example, with reference to the accompanying drawings in which:

FIG. 1 is a schematic overview of an interventional radiological procedure using the embodiment of the invention;

FIG. 2a illustrates a catheter assembly with sensors applied to the catheter with FIG. 2b illustrating a guide wire for a catheter including a plurality of sensors;

FIG. 2c illustrates a method of embedding a sensor to an external wall and of a catheter;

FIG. 2d illustrates a sensor being mounted at a tip of the catheter;

FIG. 2e illustrates a sensor being mounted at a tip of the guide wire;

FIG. 3 illustrates the forces on a catheter as this is fed through a blood vessel;

FIG. 4 is a schematic diagram of a haptic feedback apparatus;

FIG. 5 and FIG. 6 illustrate the forces on a catheter, used in calculation of control signals for the haptic feedback apparatus of FIG. 4;

FIG. 7 illustrates a detailed block diagram of a signal processing and logic control center;

FIG. 8 illustrates a deformation sensor in the form of a microgyroscope;

FIG. 9 shows a side view of the microgyroscope of FIG. 8 mounted on a glass substrate;

FIG. 10 is a block diagram depicting a processing circuit connected to the microgyroscope of FIG. 8;

FIG. 11 depicts how micro-wires are used to transmit signals from the sensors attached to the catheter of FIG. 2a to a signal processing and logic control unit for further processing;

FIG. 12 illustrates a feedback loop used in the haptic feedback apparatus of FIG. 4;

FIG. 13 shows how detection of the constraint forces in the catheter determines the shape of a catheter;

FIG. 14 is a block diagram depicting an overview of the different components of the first embodiment of the invention; and

FIG. 15 illustrates a variation of one of the sensors of FIG. 2.

### DETAILED DESCRIPTION OF THE PREFERRED EMBODIMENT

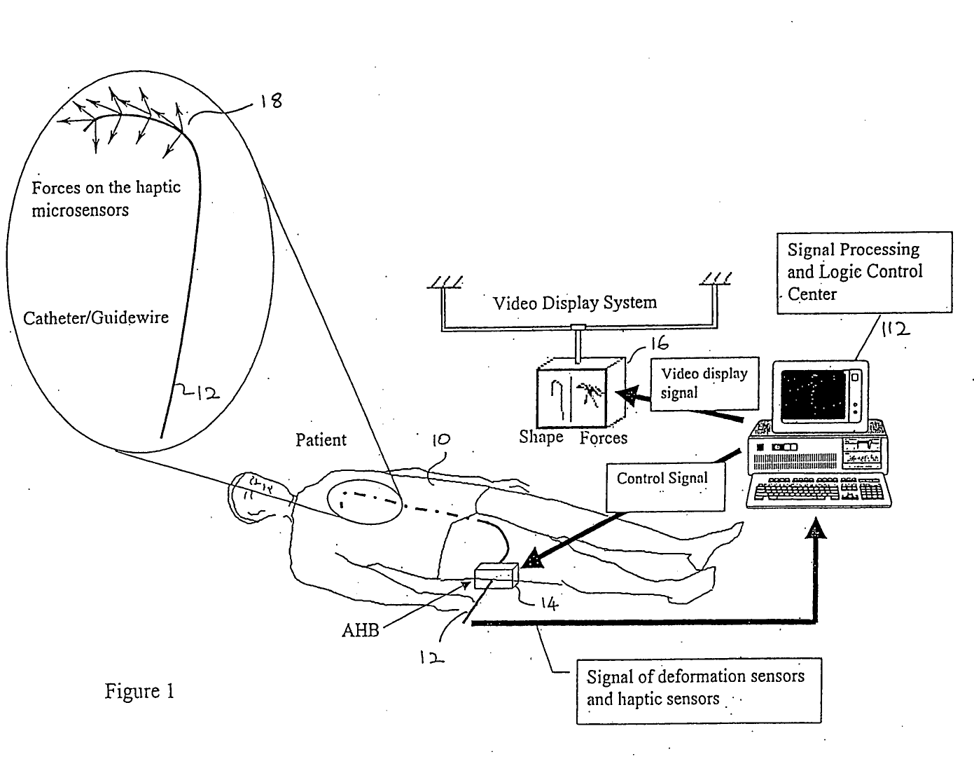

With reference to FIG. 1, a general overview of an interventional radiology procedure using an embodiment of the invention is illustrated. In such a procedure an interventional radiologist (a specialist medical doctor) inserts an elongate intervention device 12 such as a catheter 20, which at the entry stage is provided with a guide wire (not shown) to give the intervention device 12 a more solid structure, into a blood vessel of the patient and the device 12 is threaded through the vessel to a treatment location in the patient's body 10. The intervention device 12 is passed through a haptic feedback device 14 before entry to the patient 10. The intervention device 12 is of a special form being provided with a plurality of force sensors along its length which sense forces acting thereon. These sensors are connected to a control processor forming part of the haptic feedback device 14 which controls the operations of the device 14 and also sends data to a display 16 upon which the shape of the intervention device 12 in the patient's body and forces on the intervention device 12 can be displayed. The forces measured by the sensors are schematically illustrated in FIG. 1 by three orthogonal force vectors 18 at the locations of the sensors.

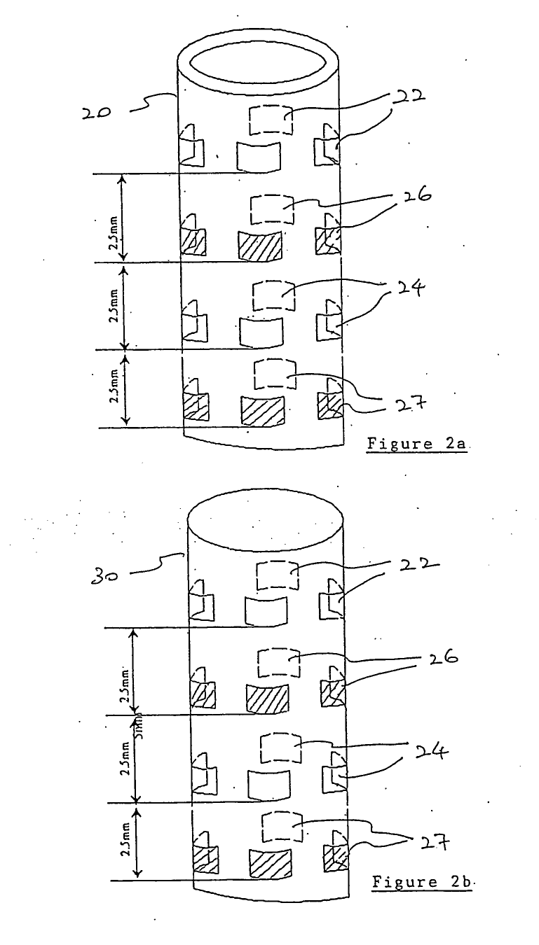

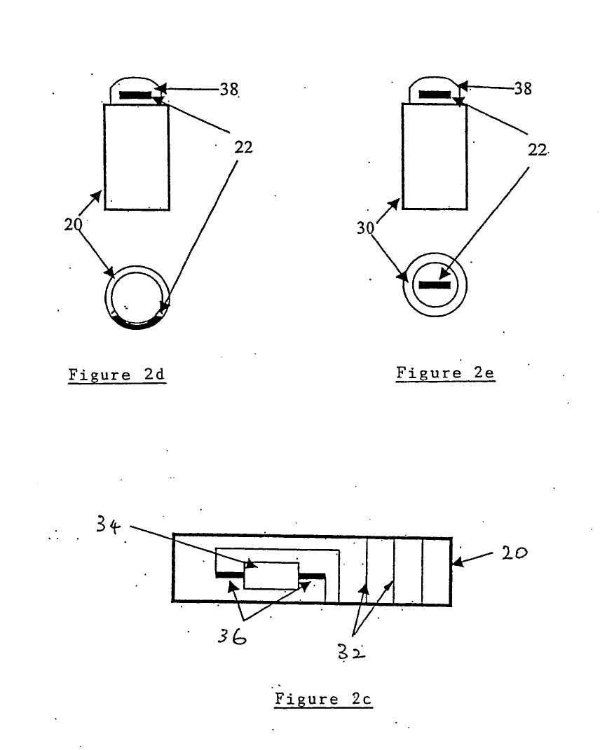

Two variations of an intervention device 12 in the form of a catheter 20 and a guide wire 30 are shown in FIGS. 2a and 2b respectively. In FIG. 2a, a catheter 20 is shown in which a plurality of haptic sensors 22, 24 are embedded in the catheter wall 23. Haptic sensors 22, 24 are of the same type with four such sensors being equi-angularly disposed in the same radial plane of the catheter 20 at intervals D of preferably 5 mm, so that each sensor 22,24 can sense three dimensional forces on the catheter 20 at that location and feedback these sensory values to the radiologist via hair-thin cables to the distal end of the catheter 20 and to the haptic feedback device 14 which acquires the data.

The second type of sensors embedded in the walls of the catheter 20 or the guide wire 30 are a plurality of deformation sensors in the form of fibre optic sensors 26,27. These deformation sensors 26,27 are disposed in the same manner as the haptic sensors 22,24 but are positioned in different radial planes so that these deformation sensors 26,27 are interleaved between the haptic sensors 22,24 as shown in FIG. 2a. Preferably, if the distance D between two sets of haptic sensors is 5 mm, then the distance between a haptic sensor 22,24 and a deformation sensor 26,27 is 2.5 mm. These deformation sensors 26,27 detect the orientation or shape of the catheter 20 so that an interventional radiologist could more intuitively navigate the catheter 20 in a human body. When the detected shape of the catheter 20 is displayed, this allows the radiologist to monitor the direction of movement of the catheter 20 as the catheter 20 is being maneuvered in the human body.

A second variation of an intervention device is shown in FIG. 2b. As noted with reference to FIG. 1, the catheter 20 when inserted may be provided with a guide wire 30 and the haptic and deformation sensors 22,24,26,27 shown in FIG. 2a can be disposed on the guide wire instead of in the catheter 20. A guide wire 30 is shown in FIG. 2b which is generally made of plastic or suitable metal and has a flexible body for steerability and is provided with haptic sensors 22, 24 and deformation sensors 26,27 disposed on the external surface of the guide wire 30.

There are two methods of embedding or connecting the micro-sensors 22,24,26,27 to the catheter 20 (it should be apparent that the methods also apply to the guidewire) and FIG. 2c shows a first method [1] in which the sensors are mounted along the catheter walls. In this method, the first step is to form wiring 32 on the outer wall to carry signals picked up by the sensors to the distal end of the catheter 20 and an electrical conductive layer is first formed on the outer catheter wall by vacuum evaporation. The wiring 32 is next patterned using excimer laser processing. In this case, as shown in FIG. 2c, the wiring 20 is helical in shape so as to conform to the shape of the catheter 20 to enhance flexibility.

After the wiring process, the conductive layer is electroplated with copper to reduce electrical resistance. Preferably, the catheter 20 is also coated with polyurethane resin to protect the wiring against bending or abrasion.

To embed each sensor to the outer catheter wall, receptacles 34 and electrodes 36 are formed. Each receptacle 34 is formed using the excimer laser process for patterning the wiring 32 and electroplating technique is used to mount a sensor within the receptacle 34. The sensor is then bonded to the receptacle using adhesive agent such as polyurethane resin. The electrodes 36 are then electrically connected to the sensor's leads.

Mounting the sensors and forming the receptacles, electrodes and wiring on the outer catheter walls has advantages, for example the catheter's diameter can be reduced and the catheter is more flexible when compared with locating the sensors and wiring along the “lumina” of the catheter 20.

A second method [2] is for mounting a sensor near or at the catheter or guide wire's tip. FIG. 2d shows side and end views of a catheter 20 with a tactile sensor 22 mounted along the rim of the catheter's tip. FIG. 2e, on the other hand, illustrates side and end views of a guide wire 30 with a tactile sensor being mounted at the tip of the guide wire. In both cases, wiring and electrode for connecting to the sensor's leads are formed using the process described earlier. Next, the sensor is attached to the tip of the catheter (or guide wire, as the case may be) using a suitable adhesive and silicon rubber 38 to form a contact portion which provides good contact with the blood vessels for enhanced sensing when the catheter/guide wire is in use.

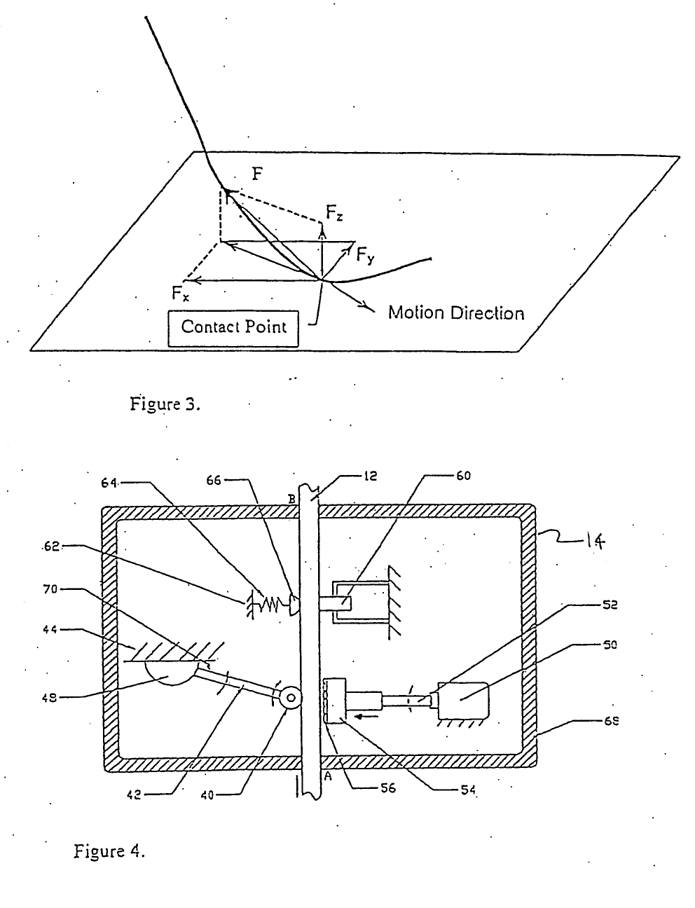

In both variations of the intervention device, the haptic sensors 22,24 are fabricated using MEMS technology such as that described in a publication “Silicon-based three axial force sensor for prosthetic applications” [3] which describes a three axial force sensor to sense normal and shear force components at a particular contact point. In the present embodiment, as shown in FIG. 3, the haptic sensor 22,24 are used to detect the forces on a catheter 20 at a particular point of contact with the internal wall of a blood vessel. The force acting on the catheter 20 at location can be resolved into three force components Fx, Fy, Fz. The forces Fx and Fy are resolved in a direction tangential to the catheter with the force component Fz being orthogonal to the plane of the forces Fx, Fy. The forces Fx and Fy, in combination, represent the frictional force between the catheter and the vessel wall, and the force Fz normal to the sliding direction causes problems of direct puncturing of the vessel walls. The vector sum of these forces (F=Fx+Fy+Fz) at a particular contact is the friction force in the opposite direction to the motion of the catheter and is used for the calculation of the haptic feedback on the catheter.

ΣF represents summation of all the friction forces F detected by each haptic sensor 22,24 along the body of the catheter/guide wire. This summation force ΣF is then processed by the Signal Processing and Logic Control Center and provided to the haptic feedback device 14 so that the effect is more readily felt by a specialist's hand. For example, if the ΣF is 5N and this force is augmented five times to 25N (denoted as F′), this means that 25N of force will be fed back to the radiologist's hand. In this way, the force sensors can thus detect the force components and to provide tactile feedback based on piezoresistive effect. The operation of the haptic feedback device 14 will be described in more detail below.

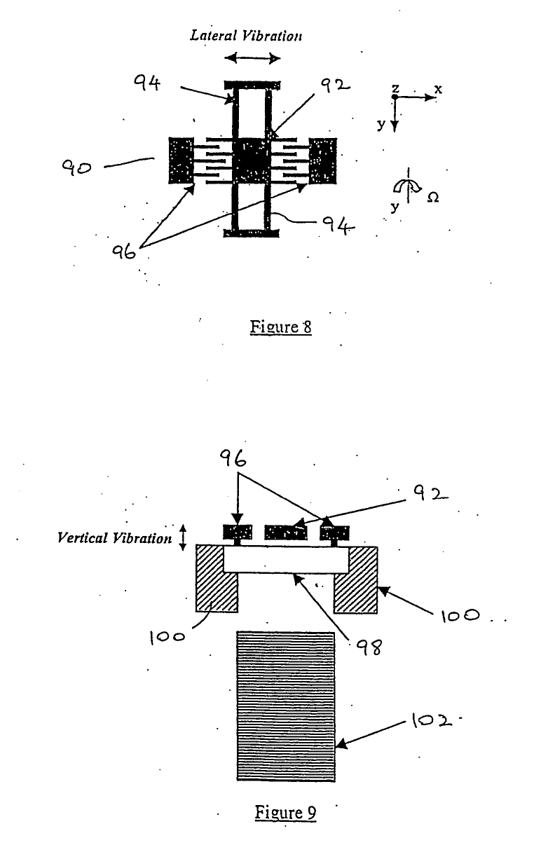

Fiber optic sensors 26,27 are suitable for use as deformation sensors because of their high resolution, accuracy and immunity to electromagnetic interference. In this embodiment, fiber optic tactile-based microgyroscopes are used as deformation sensors and FIG. 8 shows an example of a “comb-drive single mass” microgyroscope 90. The microgyroscope 90 comprises a resonator 92 (proof mass) made of silicon and suspended by four flexures or beams 94 as shown in FIG. 9. The resonator 92 is driven by electrostatic forces generated by DC and AC bias voltages across comb actuators 96 coupled to the resonator 92. When the suspended resonator 92 is vibrating, angular rate around the y-axis induces Coriolis force F_{c} which is proportional to the proof mass “m”, the vibration velocity “v” and the angular rate. This Coriolis force F_{c} causes the proof mass (resonator) to vibrate in the z direction.

To set up the microgyroscope 90 as a deformation sensor 26,27, the four beams 94 are anchored on a glass substrate 98 and FIG. 9 shows a cross-sectional view of this arrangement. The glass substrate 98 is then supported by two fiber alignment structures, preferably made of silicon. An optical fiber 102 with a suitable diameter such as 50/125 μm (core/clad) is communicatively coupled to the glass substrate 98 via a fiber optic coupler 104—fused coupler, split ratio 1:1 (not shown).

The sensing method [4] used is modulation of the light intensity since intensity of the light source changes due to the vibration of the resonator 92 located in front of the glass substrate 98 which also acts as a “fiber stopper”. This means the resonator 92 acts like a mirror reflecting the light source that is beamed to the resonator's underside and the reflected light is processed to obtain perturbation experienced by the resonator 92. Accordingly, the fiber optic coupler 104 needs to re-transmit the light to the resonator 92 and also receive back the reflected light.

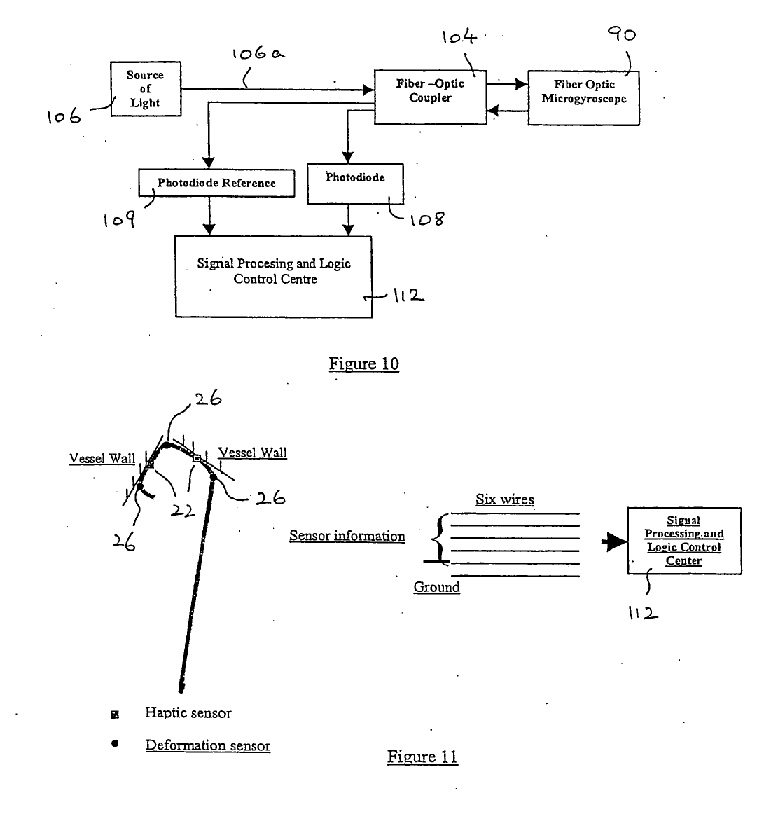

FIG. 10 is a schematic diagram showing how the reflected signal is being processed thereafter. A light source 106 such as a LED is used to provide a light beam 106a through the optical fiber 102 and to a receive port of the fiber optic coupler 104. The fiber optic coupler re-transmits the light 106a to the resonator 92 which is vibrating. The light reflected by the resonator 92 at a particular intensity in response to the vibration is transmitted back through the fiber optic coupler 104 to a detector 108 connected to the other output port of the fiber optic coupler 104. In this example, a photodiode 108 is used as the detection means to detect the light level since we are concerned with the intensity of the reflected light. A photodiode reference 109 may also be derived from the coupler 104 which can be used for calibration of the resonator 92. The detected signal level is processed by the Signal Processing and Logic Control Center 112 and this includes a Signal Conditioning Circuitry 112a which comprises filtering and amplification circuits. The conditioning circuitry 112a filters unwanted noise from the detected signal level and, if need be, amplifies the signal so that the detected signal is suitable for acquisition by the Data Acquisition Unit 112b. This unit can use normal data acquisition circuits available off the shelf such as that from National Instruments. The acquired data is then provided to a computing unit 112c for translating the data into amount of displacement of the resonator 92. As mentioned earlier, there is a plurality of deformation sensors 26,27 disposed along the length of the catheter 20 and displacement information from these sensors 26,27 provide the shape of the catheter 20. As shown in FIG. 11, when the displacement information of three of the deformation sensors 26,27 are obtained, the Signal Processing and Logic Center 112 can use known mathematical tools such as line segmentation, circle segmentation or splines (requires displacement information of at least four points), to determine the shape of the catheter 20.

After obtaining the shape information, a real-time graphics representation of the catheter 20 can be displayed on a monitor, for example, using one of several known graphics display rendering methods such as centerline model, wireframe model, surface rendered model or volume rendered model.

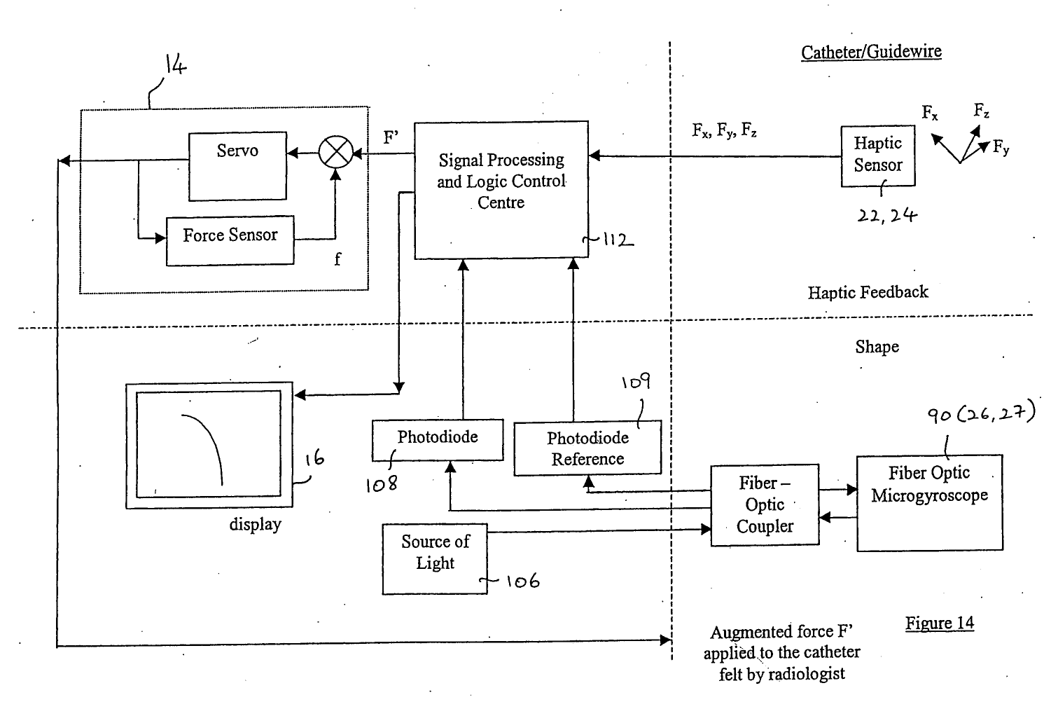

In this way, the fiber optic sensors 26,27 disposed along the length of the catheter 20 provide local deformation information which is used to determine and display the shape of the catheter. This is represented schematically in FIG. 14. This allows the radiologist to observe the orientation of the catheter 20 in the event that the tip of the catheter 20 bends backwards which causes the catheter to move in the opposite direction. Without the shape information, it may not be possible to detect this scenario if the radiologist does not feel any obstruction when inserting the catheter 20.

In addition, the radiologist when maneuvering the catheter 20 will be able to monitor the orientation (shape) of the catheter 20 when, for example, the catheter 20 turns left or right branching into an auxiliary blood vessel, and thus the interventional specialist can observe this through a tactile video display 16 (See FIG. 1).

Wiring formed on the outer catheter walls transmit signals (detected forces) picked up by the sensors 22,24,26,27 for further processing to the Signal Processing and Logic Control Centre 112. Alternatively, in the case of the fiber optic microgyroscope, fiber optic micro cables may be used to carry such information from the catheter 20. FIG. 11 shows a simplified sensor arrangement in a catheter which has two haptic sensors 22,24 and three deformation sensors 26,27 disposed along a particular length. To send signals from the sensor locations to the signal conditioning circuitry or directly to the logic and control center 112, each sensor is provided with a wire or cable. In this case, there is a total of six wires/cables with an additional wire for a ground signal.

Having described the deformation sensors 26,27 in detail, the haptic sensors 22,24 will now be described together with how the remote forces detected by the sensors 22,23 are felt by the radiologist.

In FIGS. 2a and 2b, only a small section of the catheter/guide wire is shown but in practice the sensors 22, 24 (or 32, 34) will be repeated along the length of the catheter 20 to provide force information along the whole length of the catheter 20 as this is inserted into the blood vessel of a patient. In addition, a haptic sensor (see FIG. 2d) is preferably provided at the tip of the catheter 20 to detect the force at the tip of the catheter 20 which can be used to determine the rupturing force against the wall of a blood vessel [5].

It is very important for the radiologist to be able to feel these forces since they are indicative of potential damage to the blood vessel wall. However, the actual magnitude of these forces is very small and in the described embodiment the sensors are used in two ways, firstly to display the force information for the specialist's benefit (as in the deformation sensors 26,27) and also to provide augmented haptic feedback (as in the haptic sensors 22,24) so that the specialist can feel the nature of the forces more clearly.

To allow the specialist to feel the forces more clearly, the frictional forces F detected by the sensors are processed by the signal conditioning circuit 112a, data acquisition unit 112b and the computing unit 112c. The computing unit 112c calculates the augmented force F′ and generate a signal to drive the haptic feedback device 14. This also allows the radiologist to adjust the amount of amplification via the computing unit to adapt to the feel of different radiologists.

Further, the frictional forces can be transformed into images to provide a visual image/picture of forces for the radiologist to better visualize the forces. (see for example, publication at http://page.inf.fu-berlin.de/˜kurze/publications/chi_{—}97/mk-hapt.htm). Thus, in addition to the haptic feel, the forces can also be displayed on a display.

The haptic feedback device 14 of FIG. 1 is shown in more detail in FIG. 4. The catheter 20 (guide wire) is fed through the middle of the device 14 and several control mechanisms act on the catheter 20 to apply an augmented force on the catheter 20 as the catheter 20 passes through the body. On one side of the catheter 20 a reaction device is provided which includes a first wheeled encoder 40 connected to a shaft 42. The other end of the shaft 42 is connected to a stationary support 44 via a ball and socket joint 48. Biasing means 70 in the form of a spring is included, as shown in FIG. 4, to ensure contact between the first wheeled encoder 40 and the catheter 20. The first wheeled encoder 40 is used to monitor and track the linear movement of the catheter 20. When the tip of the catheter 20 passes through the first wheeled encoder 40, an initial encoder value is initialized which corresponds to the tip of the catheter 20. When the catheter 20 advances through the haptic feedback device 14 and into the vessel, the encoder value changes which is proportionate to the amount the catheter's tip has advanced pass the first wheeled encoder 40 and into the blood vessel. In this way, the length of the catheter 20 that is inserted into the human body can be calculated.

On the other side of the catheter 20 a force supplying device is provided which includes a servo motor 50 connected to a shaft 52. At the other end of the shaft 52, a gear box 54 is provided which converts the rotational motion of the shaft 52 into a translational motion towards or away from the catheter 20. The gear box 54 and the shaft 52 may be a “rack and pinion” structure to translate the rotational force of the shaft into the required translational motion. The part of the gear box 54 which engages the catheter 20 is provided with a surface 56 made of resilient material, such as rubber, to apply a variable force normal to the catheter's length direction on the catheter 20. Other suitable surfaces may be used which provides such a resilient force.

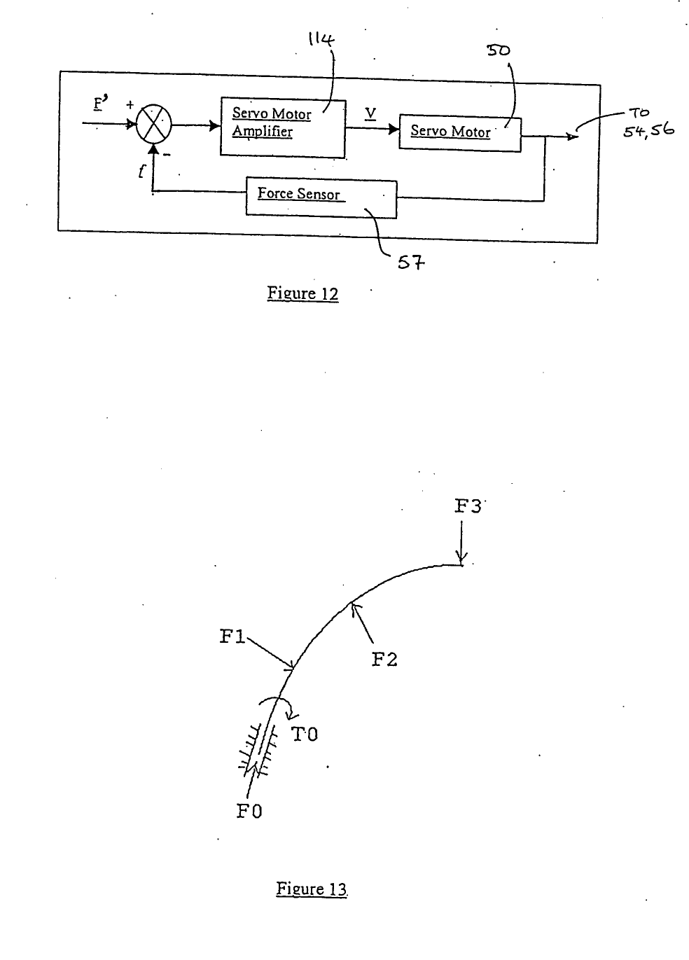

A force sensor 57 (see FIG. 12) is embedded within the surface 56 to detect the amount of pressure “P” being asserted on the catheter 20. An example of such a force sensor is a “Single Axis Force Sensor” from “Applied Robotics” (www.arobotics.com/forcesensors.htm). Let's assume that the contact between the catheter 20 and the surface 56 creates a friction coefficient of “k”. A friction force “f” acting on the catheter 20 is then calculated as “f=kP”. In this way, it is possible to detect the amount of force that is to be applied on the catheter 20. However, to control this force “f” (applied by the servo motor 50) in relation to the augmented force F′ (as derived from the sensors 22,24 embedded in the catheter wall) a feedback loop can be used and an example of which is shown in FIG. 12.

As mentioned earlier, the augmented force F′ is provided by the computing unit 112c of the Signal Processing and Logic Control Center 112 and in this embodiment since a servo motor 50 is used to drive the shaft 52, the augmented force F′ may be in the form of a square wave to control the servo motor 50. It is apparent that this can be realised in a number of ways.

If the augmented force F′>f, a servo motor amplifier 114 increases the amount of voltage V to the servo motor 50 to drive the gear box 54 so that the friction force “f” applied on the catheter 20 increases. The increased in friction force “f” acts on the catheter 20 to resist the advance of the catheter 20 and thus the radiologist experiences increased resistance. On the other hand, if F′<f, the amplifier reduces the voltage V to the servo motor so that the friction force “f” acting on the catheter 20 is reduced. This allows the catheter 20 to advance with greater ease. If F′=f, then voltage V will be maintained and f will not change. In this way, the correct amount of force “f” is applied on the catheter 20 without causing any damage to the vessel walls. Preferably, this force “f” (and the augmented force F′) is computed continuously so that the variable force “f” is continuously updated to ensure the correct amount of force is applied onto the catheter 20. Alternatively, this variable force “f” may be computed only at regular intervals when used in a less critical application such as in a simulation experiment.

A second wheeled encoder 60 is also provided in the haptic feedback device 14 which encodes the rotational movement of the catheter 20. On the other side of the catheter 20 a biasing arrangement comprising a support 62, a spring 64 coupled to the support 62 at one end and a contact member 66 resiliently attached to the other end of the spring 64, is used to ensure contact between the catheter 20 and the second wheeled encoder 60 as the catheter 20 advances through the haptic feedback device 14. In this embodiment, the contact member 66 is in the form of a ball which rotates freely and is biased by the spring 64 so that the catheter 20 is always in contact with the second wheeled encoder 60 to track the rotational movement of the catheter 20. Similar to the first wheeled encoder 40, the second wheeled encoder 60 is initialized to a reference position of the catheter 20 so that any subsequent rotational movement may be registered.

In use, if the catheter 20 experiences increased friction as sensed by the sensors 22, 24 when the catheter 20 travels through a blood vessel, the haptic feedback device 14 acts on the catheter 20 to amplify that force to make it more noticeable to the specialist. Specifically, with an increase in sensed resistance, the device 14 causes the surface 56 to approach the catheter 20 which, due to increased friction between the catheter 20, wheel 40 and the surface 56 increases the degree of resistance that the specialist will feel when feeding the catheter into the blood vessel. In this way, the specialist feels an augmented force F′ when operating the catheter 20. Similarly, if the catheter starts to twist in the body, the rotation is picked up by the second wheel encoder 60 and this rotation can be recorded. For example, when the catheter 20 reaches a branch, the user of the catheter may need to rotate the catheter to change the direction of motion. By recording this information, the direction of the catheter can be obtained.

If the specialist wants to pull back the catheter/guide wire 20,30 force sensors detect a decrease in pressure and feeds this change to the servo motor 50 which rotates the shaft 52 clockwise thus releasing the normal force “f” on the catheter 20.

FIG. 14 depicts an overall block diagram according to the first embodiment and illustrates how the various components are connected to each other. The haptic sensors 22,24 are arranged to determine the three dimensional frictional forces acting on the catheter 20 as the catheter 20 navigates through a blood vessel. This information is processed by the control center 112 which provides an augmented force F′ via the feedback control device 14 to the body of the catheter 20. In this way, as the radiologist pushes or rotates the catheter 20 into the blood vessel, the remote force picked up by the haptic sensors 22,24 are amplified and provided to the catheter 20 so that the radiologist can interpret or feel more clearly any possible obstruction to alleviate rupturing of the blood vessel. This allows the radiologist to better “localise” the catheter's movement within the blood vessel. The control center 112 also obtains displacement information from the fiber optic deformation sensors 26,27 which can be translated into shape of the catheter for visual observation by the radiologist.

In combination, the shape of the catheter and the haptic feedback helps the radiologist to perform the catherisation procedure with greater accuracy and convenience alleviating a need to take X-ray images of the patient whenever there is an indication of obstruction of the catheter.

The first embodiment of the present invention may also be used for simulation training in which the blood vessel or vasculature is simulated virtually. For this purpose, the human subject may be replaced by a FEM model such as that disclosed in PCT/SG01/00111, the contents of which are incorporated herein by reference. However, unlike a real patient, the sensors 22,24,26,27 on the catheter 20 will not detect the actual forces and thus the haptic feedback force needs to be calculated and provided to the specialist navigating the catheter via the haptic feedback device 14 in order to provide a “life-like” catheterisation procedure.

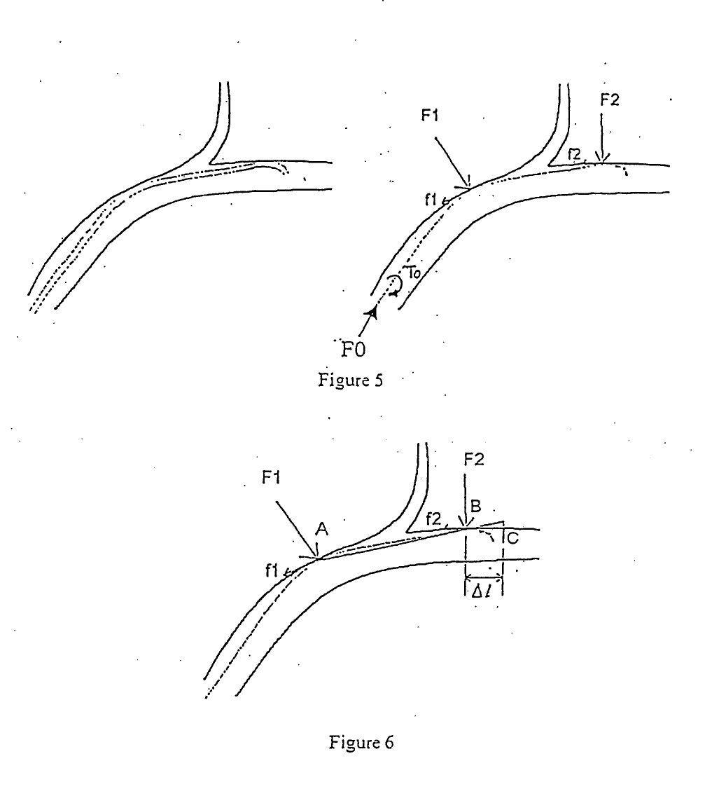

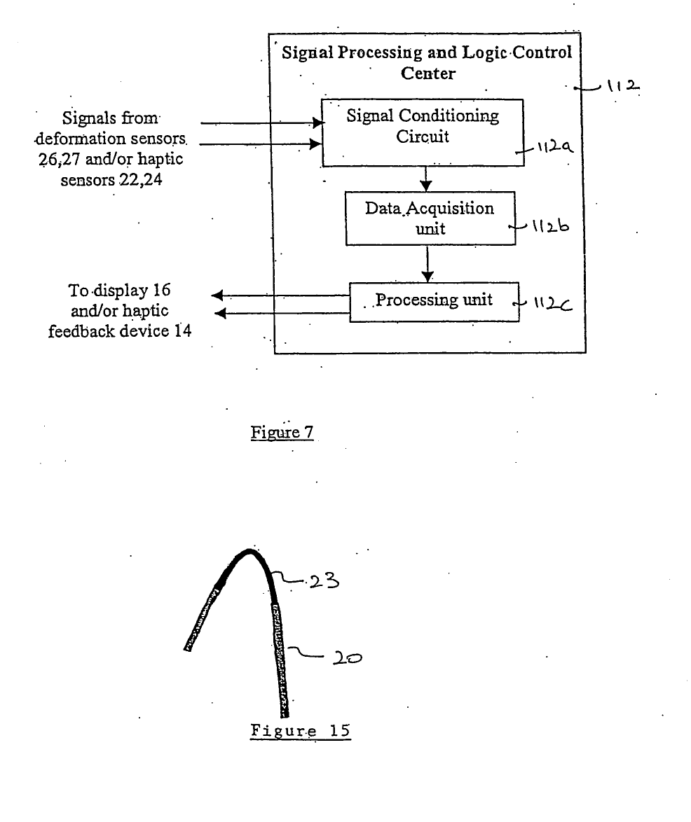

Instead of detecting the constraint forces Fx, Fy and Fz, the simulation model calculates and provides simulated constraint forces F1,F2 for display and this will be described with reference to FIGS. 5 and 6. Vectors F1 and F2 are constraint forces from two contacting points (in actual simulation there would be many of these since in practice there are also a plurality of constraint forces). If the catheter is moving, friction forces f1, f2 in FIG. 5 are also generated. The force feedback F0 and the twisting torque T0 will be equal to the sum of the constraint forces and frictional forces from all of the contact points.

By measuring the force and torque on the catheter 20 along its length, the feedback force and torque F0, T0 can be estimated and for processing by the signal processing and logic unit 112 which subsequently provides amplified versions of the force via the haptic feedback device 14 to the catheter 14 in the same way as a real procedure.

The detection of the simulated forces experienced by the catheter 20 are computed from a computer model of a simulated vasculature and the computation method and processing are known as explained in “Real-Time Interactive Simulator for Percutaneous Coronary Revascularization Procedures” [8]. Thus, this will not be further elaborated here.

Similar to a real procedure, when the radiologist interacts in the virtual environment, the forces experienced by the radiologist may be displayed using for example haptics rendering as explained in “Dynamic Models for Haptic Rendering Systems” [9].

The third embodiment of the invention envisages using the invention according to the second embodiment to assess the skills of a practitioner such as an intervention radiologist.

In a catheterisation procedure, such as peripheral, cardiac or neuro catherisation, the success rate may depend on the skills of the interventional radiologist. Complications may result from such procedures if the vascular walls are damaged which may be caused by the use of excessive force when steering or navigating the catheter through the vessels.

For the third embodiment, a database would first be set up which contains a permissible range of forces that may be exerted on a specific region of an artery or a blood vessel without causing damage. Such biomechanical measurements may be obtained from prior literatures [6],[7] and an example of such a database is shown below: a

TABLE 1

| | Density | Bulk modulus | Shear modulus | Poisson |
|---|---|---|---|---|
| Tissue | (ton/m3) | (Gpa) | (Gpa) | ratio |
| Bridging veins | 1.133 | EA = 1.9N | | 0.45 |

The maximum force or stress that the vein (or vessel wall) can sustain can then be determined using finite element analysis. An example is to model the vein using eight nodes brick elements and assuming a linear model, the minimum force required to damage the vein can be estimated using a finite element solver such as ABACUS. In this example, the above vein parameter translate to a force of less than IN that can be exerted on the particular vein without causing damage.

As described in the second embodiment, the haptic sensors 22,24 of the catheter 20 are arranged to measure the various simulated forces acting on the catheter at that point. Therefore, these forces can be determined and compared with the data in the database above for the skills assessment. From the comparison, a score may then be assigned depending on how well the radiologist manages to control the catheter such that the force exerted on that portion of the vessel is within that of the collected range in the database.

In a fourth embodiment, the invention may be used as a tool for validation of catherisation simulators.

Catherisation simulators are important in the training of interventional specialists, particularly their “motor skills”, which is the interventional specialist's ability to manipulate or guide the catheter/guidewire to the target area of the patient's body. A typical software simulator measures the amount of contrast dye and X-ray used and the time taken for a targeted navigation. Such simulators also compute the forces experienced by the catheter during the procedure and feedback such forces to a force feedback device and to an interventional radiologist participating in the simulation. The accuracy of the computation and the resultant tactile force is critical and thus there is a need to validate these parameters.

The fourth embodiment of the invention proposes the use of the force measuring sensors and the catheter to validate such a catheterisation simulation. An in-vivo experiment may be conducted using human subjects so that forces at various points of an artery or blood vessel may be measured and recorded. These forces are then compared with the corresponding computed forces from the simulator. If, for example, the forces experienced by the tip of the catheter at a particular section along a artery have a scalar quantity of 0.002N, then the measured forces at the same section should have approximately the same value.

The described embodiments should not be construed as limitative. For example, in FIGS. 2a and 2b, four haptic sensors are used to detect three dimensional force acting on a particular point. Alternatively, more sensors can be arranged to cover the whole surface of the catheter which would improve the accuracy of the measurement. However, this will make the catheter more complex and may increase the difficulty of manufacturing.

In another variation, other suitable light source may be use, for example a laser diode, for use by the microgyroscope. Other parameters of the reflected light can also be measured to understand the perturbation experienced by the resonator other than measuring the intensity of the reflected light.

The described embodiment uses a catheter 20 as an example but it will be apparant that what is described can similarly be applied to a guide wire. In the described embodiment, the catheter 20 is arranged to be inserted into a human subject but the same interventional procedure may be used on an animal body.

Instead of providing a plurality of deformation sensors along the catheter 20, a “distributed sensor” 23 such as that shown in FIG. 15 may be used to determine the shape of the catheter 20 at that location. The distributed sensor 23 includes a plurality of micro sensors and together these micro sensors determine the deformation of the catheter 20 similar to that described earlier.

### REFERENCES

[1] Satoshi Nakagawa, Hitoshi Ozasa, and Hiroshi Misawa; Development of Micro-Wiring on the Outer Wall of a Catheter; IEEE Proceedings of the Sixth International Symposium on Micro Machine and Human Science, 1995, pp. 137-143.
[2] Hironobu Takizawa, Hiroshi Tosaka, Ryo Ohta, Shinji Kaneko, and Yasuhiro Ueda; Development of a Microfine Active Bending Catheter Equipped with MIF Tactile Sensors; Proceedings of the Twelfth IEEE International Conference on MEMS (MEMS'99), 1999, pp. 412-417.
[3] L. Beccai, S. Roccella, A. Arena, A. Menciassi, M. C. Carrozza, P. Dario; Silicon-Based Three Axial Force Sensor for Prosthetic Applications; The 7^{th} National Conference on Sensors and Microsystems, Bologna, Feb. 4-6, 2002.
[4] O. Tohyama, S. Maeda, and H. Itoh; Fiber-Optic Tactile Microsensor for Detecting the Position of the Tip of a Fiberscope; IEEE Journal of Selected Topics in Quantum Electronics, Vol. 5, No. 1, pp. 115-118, 1999.
[5] Masayoshi Esashi; Microsensors and Microactuators for biomedical application; Faculty of Engineering, Tohoku University Aza Aoba Aramaki Aoba-ku Sendai 980-77, Japan.
[6] Warren C Young; Roark's Formulas for Stress & Strain; 6^{th} Edition, McGraw-Hill, Inc. 1989.
[7] Hiroshi Yamada: Strength of Biomedical Materials; The Williams & Wilkins Company Baltimore, 1970.
[8] Yaoping Wang, Cheekong Chui, Honglip Lim, Yiyu Cai and Koonhou Mak; “Real-Time Interactive Simulator for Percutaneous Coronary Revascularization Procedures”; Computer Aided Surgery 3:211-227 (1998)
[9] Diego Ruspini and Oussama Khatib; “Dynamic Models for Haptic Rendering Systems”; Advances in Robot Kinematics: ARK'98, June 1998, Strobl/Salzburg, Austria, pp 523-532.

1. Haptic feedback apparatus comprising:
force application means arranged to apply a force to an elongate intervention device,
control means arranged to control the force applied to the intervention device by the force application means, the control means being connected to at least one sensor arranged to sense a remote force on the intervention device and the control means being arranged to calculate the applied force in accordance with the remote force, the applied force being an amplification of the remote force,
wherein the force application means comprises a resilient member arranged to apply the said force to the intervention device, and
wherein the apparatus further comprises a sensor arranged to detect frictional force between the resilient member and the intervention device.

2. Haptic feedback apparatus according to claim 1, wherein the detected frictional force is used to control the amount of applied force.

3. Haptic feedback apparatus according to claim 1, further comprising means for tracking the rotational movement of the intervention device.

4. Haptic feedback apparatus according to claim 1, further comprising means for tracking the linear movement of the intervention device.

5. Haptic feedback apparatus according to claim 1, further comprising means for comparing the remote force with a reference force.

6. Haptic feedback apparatus according to claim 1, wherein the intervention device is suitable for insertion into a simulated human model.

7. Haptic feedback apparatus according to claim 6, wherein the remote force is generated using computer simulation.

8. Haptic feedback apparatus according to claim 7, wherein the intervention device is operable to be inserted into a human subject.

9. Haptic feedback apparatus according to claim 8, wherein the at least one sensor is disposed near or at a tip of the intervention device.

10. Haptic feedback apparatus according to claim 7, further comprising a plurality of sensors disposed along the length of the intervention device and the control means is connected to each of the plurality of sensors.

11-36. (canceled)
